(19) United States
(12) Patent Application Publication
(10) Pub. No.: US 2023/0044120 A1
(43) Pub. Date: Feb. 9, 2023

(54) ULTRASONIC BLOOD PERFUSION IMAGING METHOD AND APPARATUS FOR SINGLE VESSEL

(71) Applicant: Nanjing Transcend Vivoscope Bio-Technology Co., LTD, Nanjing (CN)

(72) Inventors: Jian AN, Nanjing (CN); Feihong DONG, Nanjing (CN); Jiabin ZHANG, Nanjing (CN); Jue ZHANG, Nanjing (CN)

(73) Assignee: Nanjing Transcend Vivoscope Bio-Technology Co., LTD, Nanjing (CN)

(21) Appl. No.: 17/970,905

(22) Filed: Oct. 21, 2022

Related U.S. Application Data
(63) Continuation of application No. PCT/CN2021/077466, filed on Feb. 23, 2021.

(30) Foreign Application Priority Data
Jun. 24, 2020 (CN) 202010590188.5

(51) Int. Cl. A61B 8/06; A61B 8/08

(52) U.S. Cl. CPC A61B 8/06; A61B 8/0891; A61B 8/48

(57) ABSTRACT

Disclosed is an ultrasonic blood perfusion imaging method for a single blood vessel, comprising: setting an ultrasound focusing label point in a blood vessel contour of a blood vessel to be measured in a region to be measured; obtaining a preactivated ultrasound image of the region to be measured when an ultrasound contrast agent is in an inactive state; activating the ultrasound contrast agent; obtaining an activated ultrasound image of the region to be measured when the ultrasound contrast agent is in an activated state; obtaining an activation map of the ultrasound contrast agent in the blood vessel to be measured; and obtaining a blood flow perfusion distribution map of the blood vessel to be measured. The ultrasound contrast agent is activated at the ultrasound focusing label point causing liquid-to-gas conversion, and the ultrasound signal changes from dark to bright.

### CROSS-REFERENCE TO RELATED APPLICATIONS

The present application is a continuation of International Application No. PCT/CN2021/077466, filed on Feb. 23, 2021, which claims priority to Chinese Patent Application No. 202010590188.5, filed on Jun. 24, 2020. All applications are incorporated herein by reference in their entireties.

### TECHNICAL FIELD

This application relates to the technical field of ultrasound imaging, and in particular, to an ultrasonic blood perfusion imaging method for a single blood vessel, an ultrasonic blood perfusion imaging apparatus for a single blood vessel, an electronic device, and a computer-readable storage medium.

### BACKGROUND

With the development of ultrasound technology, Contrast Enhanced Ultrasound (CEUS) has been widely used to measure blood flow and blood flow perfusion in tissues, organs and tumors due to its advantages of non-invasiveness and non-radiation. However, when the traditional microbubble contrast agent enters a region to be measured, it shows an enhanced effect on all blood vessels, so it is impossible to observe the blood flow perfusion of a specific blood vessel.

### SUMMARY

In view of this, the embodiments of this application provide an ultrasonic blood perfusion imaging method for a single blood vessel, an ultrasonic blood perfusion imaging apparatus for a single blood vessel, and a computer-readable storage medium, so as to solve the technical problem in the prior art that the blood vessels inside the region to be measured are all enhanced together so that the blood flow perfusion of a specific blood vessel cannot be observed.

According to an aspect of this application, an embodiment of this application provides an ultrasonic blood perfusion imaging method for a single blood vessel, comprising: setting an ultrasound focusing label point within a blood vessel contour of a blood vessel to be measured in a region to be measured; obtaining a preactivated ultrasound image of the region to be measured when an ultrasound contrast agent is in an inactive state, the ultrasound contrast agent comprising a plurality of nano-droplets, and the nano-droplets being in a nano-droplet state when the ultrasound contrast agent is in an inactive state; activating the ultrasound contrast agent based on the ultrasound focusing label point; obtaining an activated ultrasound image of the region to be measured when the ultrasound contrast agent is in an activated state; obtaining an activation map of the ultrasound contrast agent in the blood vessel to be measured based on the preactivated ultrasound image and the activated ultrasound image, the nano-droplets being in a microbubble gas state when the ultrasound contrast agent is in an activated state; and obtaining a blood flow perfusion distribution map of the blood vessel to be measured based on a plurality of activation maps arranged in time sequence.

According to another aspect of this application, an embodiment of this application provides an ultrasonic blood perfusion imaging apparatus for a single blood vessel comprising: a labeling module, configured to set an ultrasound focusing label point within a blood vessel contour of a blood vessel to be measured in a region to be measured; a preactivated ultrasound image obtaining module, configured to obtain a preactivated ultrasound image of the region to be measured when an ultrasound contrast agent is in an inactive state, the ultrasound contrast agent including a plurality of nano-droplets, and the nano-droplets in the ultrasound contrast agent being in a nano-droplet state when the ultrasound contrast agent is in an inactive state; an activating module, configured to activate the ultrasound contrast agent based on the ultrasound focusing label point; an activated ultrasound image obtaining module, configured to obtain an activated ultrasound image of the region to be measured when the ultrasound contrast agent is in an activated state, the nano-droplets being in a microbubble gas state when the ultrasound contrast agent is in an activated state; an activation map calculating module, configured to obtain an activation map of the ultrasound contrast agent in the blood vessel to be measured based on the preactivated ultrasound image and the activated ultrasound image; and a distribution map obtaining module, configured to obtain a blood flow perfusion distribution map of the blood vessel to be measured based on a plurality of the activation maps arranged in time sequence.

According to yet another aspect of this application, an embodiment of this application provides an electronic device, including: a processor; a memory; and computer program instructions stored in the memory, the computer program instructions, when executed by the processor, causing the processor to perform any of the above-mentioned ultrasonic blood perfusion imaging method for a single blood vessel.

According to yet another aspect of this application, an embodiment of this application provides a computer-readable storage medium, where computer program instructions are stored in the computer-readable storage medium, and the computer program instructions, when executed by the processor, causing the processor to perform any of the above-mentioned ultrasonic blood perfusion imaging method for a single blood vessel.

In the ultrasonic blood perfusion imaging method for a single blood vessel provided by embodiments of this application, the ultrasound focusing label point is set within the blood vessel contour of the blood vessel to be measured in the region to be measured. The ultrasound contrast agent is activated at the ultrasound focusing label point, so that the ultrasound contrast agent changes from the inactive state to the activated state, making the nano-droplets undergo the liquid-to-gas conversion from the nano-droplet state to the microbubble gas state, realizing the conversion of the ultrasound signal from dark to light. The preactivated ultrasound image obtained under the dark signal is used as a background. Based on the activated ultrasound image obtained under the bright signal, the activation map of the ultrasound contrast agent in the blood vessel to be measured is obtained. The blood flow perfusion distribution map of the blood vessel to be measured is obtained based on a plurality of the activation maps arranged in time sequence.

Because the traditional microbubble contrast agent is always in the microbubble state and the ultrasound signal is always a bright signal, although the blood vessels can be distinguished from the surrounding tissues, the blood flow perfusion of a specific blood vessel cannot be observed when all the blood vessels show an enhanced effect. However, in the embodiments of this application, an ultrasound focusing label point is set in the blood vessel to be measured, so that the ultrasound contrast agent in the blood vessel to be measured at the labeled point can be converted from a liquid state to a gas state, making the labeled ultrasound signal in the blood vessel to be measured change from dark to bright while other unlabeled blood vessels are still under the dark signal. The difference between the ultrasound images under the bright and dark signals is used to more effectively highlight the blood flow perfusion situation in the labeled blood vessels to be measured.

### BRIEF DESCRIPTION OF THE DRAWINGS

FIG. 1 is a schematic flowchart of an ultrasonic blood perfusion imaging method for a single blood vessel according to an embodiment of this application.

FIG. 2 is a preprocessed ultrasound image of a region to be measured according to the embodiment of this application.

FIG. 3 is a blood flow perfusion distribution image of a blood vessel to be measured according to the embodiment of this application.

FIG. 4 is a schematic flowchart of setting an ultrasound focusing label point in the ultrasonic blood perfusion imaging method for a single blood vessel according to the embodiment of this application.

FIG. 5 is a schematic flowchart of obtaining a preprocessed ultrasound image of a region to be measured in the ultrasonic blood perfusion imaging method for a single blood vessel according to the embodiment of this application.

FIG. 6 is a schematic flowchart of obtaining a preactivated ultrasound image of the region to be measured in the ultrasonic blood perfusion imaging method for a single blood vessel according to the embodiment of this application.

FIG. 7 is a schematic flowchart of activating an ultrasound contrast agent in the ultrasonic blood perfusion imaging method for a single blood vessel according to the embodiment of this application.

FIG. 8 is a schematic flowchart of obtaining an activated ultrasound image of the region to be measured in the ultrasonic blood perfusion imaging method for a single blood vessel according to the embodiment of this application.

FIG. 9 is a schematic flowchart of obtaining an activation map of an ultrasound contrast agent in a blood vessel to be measured in the ultrasonic blood perfusion imaging method for a single blood vessel according to an embodiment of this application.

FIG. 10 is a schematic flowchart of obtaining a blood flow perfusion distribution map of a blood vessel to be measured based on a plurality of activation maps arranged in time sequence in the ultrasonic blood perfusion imaging method for a single blood vessel according to an embodiment of this application.

FIG. 11 is a schematic flowchart of an ultrasonic blood perfusion imaging method for a single blood vessel according to an embodiment of this application.

FIG. 12 is a schematic diagram showing positions of a first nano-droplet in activated ultrasound images at different times according to an embodiment of this application.

FIG. 13a is a schematic flowchart of calculating a first flow velocity of a first nano-droplet in the blood vessel to be measured in the ultrasonic blood perfusion imaging method for a single blood vessel according to an embodiment of this application.

FIG. 13b is a blood flow velocity diagram in a blood vessel to be measured according to an embodiment of this application.

FIG. 14 is a schematic flowchart of an ultrasonic blood perfusion imaging method for a single blood vessel according to an embodiment of this application.

FIG. 15 is a schematic diagram of an ultrasonic blood perfusion imaging apparatus for a single blood vessel according to an embodiment of this application.

FIG. 16 is a schematic diagram of an ultrasonic blood perfusion imaging apparatus for a single blood vessel according to an embodiment of this application.

FIG. 17 is a schematic structural diagram of an electronic device according to an embodiment of this application.

### DETAILED DESCRIPTION OF THE EMBODIMENTS

The technical solutions in the embodiments of this application will be clearly and completely described below with reference to the accompanying drawings in the embodiments of this application. Obviously, the described embodiments are only a part of the embodiments of this application, but not all of the embodiments. Based on the embodiments in this application, all other embodiments obtained by those of ordinary skill in the art without creative work fall within the protection scope of this application.

Application Overview

X-ray Digital Subtraction Angiography (DSA) is the most commonly used imaging method to evaluate vascular blood supply, but DSA requires invasive injection of a contrast agent needle into a designated blood vessel, and a strong exposure to radiation is experienced during the imaging process. Although Arterial Spin Labeling (ASL) in magnetic resonance imaging is a non-invasive and radiation-free perfusion functional imaging diagnosis method using endogenous contrast agents, a temporal resolution and a spatial resolution of ASL are both relatively low and labeling a single blood vessel is difficult to be realized.

With the development of ultrasound technology, Contrast Enhanced Ultrasound (CEUS) has been widely used to measure blood flow and blood flow perfusion in tissues, organs and tumors due to its advantages of non-invasiveness and non-radiation. However, traditional ultrasound contrast agents are micron-sized bubbles and belong to the gas state. After the traditional ultrasound contrast agent enters a region to be measured, the ultrasound signal in the ultrasound image is always a bright signal. Although blood vessels can be distinguished from surrounding tissues, it is unable to observe the blood flow perfusion situation of a specific blood vessel when an enhanced effect is shown on all the blood vessels.

At present, an ultrasonic blood perfusion imaging method for a single blood vessel is urgently needed, which can solve the difficulty of real-time observation of the blood flow perfusion characteristics of a single blood vessel under ultrasound.

In view of the above-mentioned technical problems, the basic idea of this application is to provide a ultrasonic blood perfusion imaging method for a single blood vessel. The ultrasound focusing label point is set within a blood vessel contour of a blood vessel to be measured in a region to be measured. An ultrasound contrast agent at the ultrasound focusing label point is activated, so that the ultrasound contrast agent changes from an inactive state to an activated state, making the nano-droplets undergo a liquid-to-gas conversion from a nano-droplet state to a microbubble gas state and making the ultrasound signal in the labeled blood vessel to be measured change from dark to light while other unlabeled blood vessels are still under the dark signal. The preactivated ultrasound image obtained under the dark signal is used as a background. Based on the activated ultrasound image obtained under the bright signal, the activation map of the ultrasound contrast agent in the blood vessel to be measured is obtained using the difference between the ultrasound images under the bright and the dark signal. The blood flow perfusion distribution map of the blood vessel to be measured is obtained based on the plurality of activation maps arranged in time sequence, so that the blood flow perfusion situation in the labeled blood vessel to be measured is more effectively highlighted.

Having introduced the basic principles of this application, various non-limiting embodiments of this application will be described in detail below with reference to the accompanying drawings.

Exemplary Method for Single Blood Vessel Ultrasound Perfusion Imaging

FIG. 1 is a schematic flowchart of an ultrasonic blood perfusion imaging method for a single blood vessel according to an embodiment of this application. As shown in FIG. 1, the ultrasonic blood perfusion imaging method for a single blood vessel includes the following steps.

Step S101: setting an ultrasound focusing label point within a blood vessel contour of a blood vessel to be measured in a region to be measured.

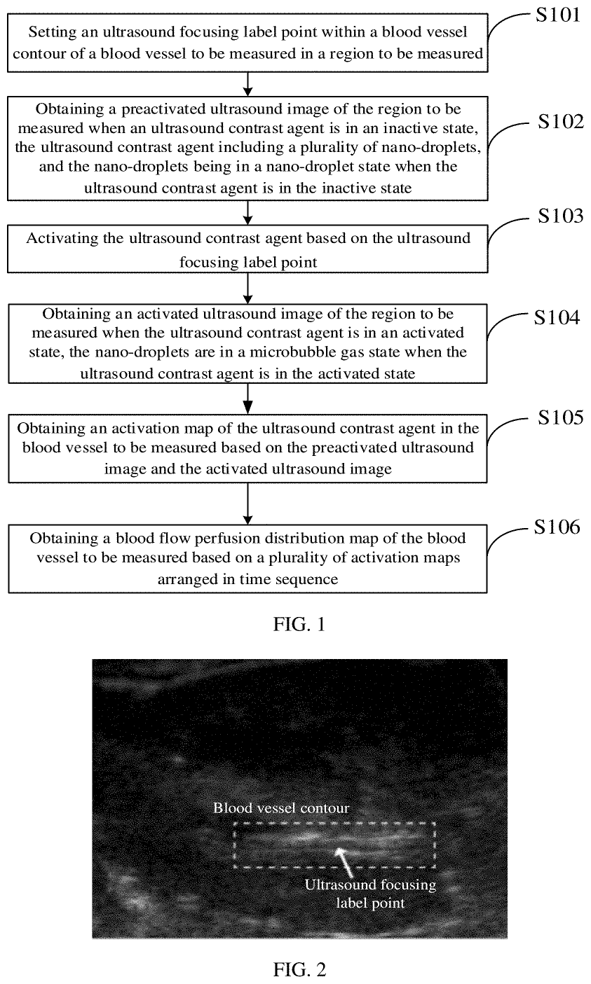

To measure a blood-vessel perfusion situation of a single blood vessel, it is necessary to use a liquid-to-gas conversion of nano-droplets in a ultrasound contrast agent to turn an ultrasound signal of a labeled blood vessel to be measured from dark to bright, in order to distinguish the ultrasound signal from a signal of other unlabeled blood vessels. It is also necessary to accurately determine an activation range, so as to ensure that the ultrasound contrast agent in the blood vessel to be measured can be activated. FIG. 2 is a preprocessed ultrasound image of the region to be measured according to the embodiment of this application. As shown in FIG. 2, according to the obtained preprocessed ultrasound image of the region to be measured, a blood vessel contour of the blood vessel to be measured in the region to be measured is manually determined, and the ultrasound focusing label point is manually set within the blood vessel contour to ensure that the ultrasound is focused into the blood vessel to be measured. A reference point for a subsequent activation of the ultrasound contrast agent in the blood vessel to be measured is provided, so as to ensure that the ultrasound contrast agent inside the blood vessel to be measured can be activated while the ultrasound contrast agent in other regions remains in an inactive state.

The specific obtaining method of the preprocessed ultrasound image of the region to be measured may be as follows: injecting the ultrasound contrast agent that can be transformed from liquid to gas; when the ultrasound contrast agent is in a preprocessing state (the ultrasound contrast agent is in the preprocessing state means that the ultrasound contrast agent stays in the liquid state, that is, when the nano-droplets in the ultrasound contrast agent are in a liquid state), detecting the region to be measured using an ultrasound probe; when a contrast enhancement signal appears, starting to collect the contrast data; and obtaining the preprocessed ultrasound image of the region to be measured. It is acceptable as long as the contrast image of the region to be measured is obtained when the ultrasound contrast agent is in the inactive state. The embodiments in this application do not limit the specific method for obtaining the preprocessed ultrasound image of the region to be measured.

Step S102: obtaining a preactivated ultrasound image of the region to be measured when the ultrasound contrast agent is in an inactive state, the ultrasound contrast agent including a plurality of nano-droplets, and the nano-droplets being in a nano-droplet state when the ultrasound contrast agent is in the inactive state.

When the ultrasound contrast agent is in the inactive state, that is, the nano-droplets in the ultrasound contrast agent are in the nano-droplet state, the preactivated ultrasound image of the region to be measured is obtained, and the preactivated ultrasound image of the region to be measured obtained under a dark signal is used as a subsequent background.

The ultrasound contrast agent includes a plurality of nano-droplets, and a concentration of the nano-droplets may be 1×10⁵ to 1×10¹¹/ml. The concentration of the nano-droplets can be adjusted according to specific application scenarios, and the embodiments of this application do not specifically limit the concentration of the nano-droplets.

Step S103: activating the ultrasound contrast agent based on the ultrasound focusing label point.

Activating refers to changing the ultrasound contrast agent from the inactive state to an activated state. Activating the ultrasound contrast agent makes the nano-droplets in the blood vessel to be measured change from a liquid state to a gas state, makes the ultrasound signal change from the dark signal to a bright signal, and makes the labeled blood vessel to be measured be under the bright signal, so that the ultrasound signal of the labeled blood vessel to be measured is made to change from dark to bright while other unlabeled blood vessels remain under the dark signal.

Step S104: obtaining an activated ultrasound image of the region to be measured when the ultrasound contrast agent is in the activated state, the nano-droplets being in a microbubble gas state when the ultrasound contrast agent is in the activated state.

After the ultrasound contrast agent is activated, the nano-droplets in the ultrasound contrast agent are in the microbubble gas state. Since the ultrasound focusing label point is set in the blood vessel contour of the blood vessel to be measured, only the nano-droplets in the labeled blood vessel to be measured are in the microbubble gas state, and the nano-droplets in other unlabeled blood vessels are in liquid state, so that the labeled blood vessels to be measured are under the bright signal, while other unlabeled blood vessels are still under the dark signal. The difference between the labeled blood vessel to be measured and other surrounding blood vessels is reflected in real time based on the activated ultrasound image of the region to be measured obtained under the bright signal.

Step S105: obtaining an activation map of the ultrasound contrast agent in the blood vessel to be measured based on the preactivated ultrasound image and the activated ultrasound image.

The preactivated ultrasound image of the region to be measured is the ultrasound image under the dark signal. the activated ultrasound image of the region to be measured is the ultrasound image with the blood vessel to be measured in the bright signal and other blood vessels in the dark signal. By a comparison between the activated ultrasound image and the preactivated ultrasound image, the activation map of the ultrasound contrast agent in the blood vessel to be measured can be obtained, that is, the activation map of the nano-droplets in the blood vessel to be measured can be obtained.

Step S106: obtaining a blood flow perfusion distribution map of the blood vessel to be measured based on a plurality of the activation maps arranged in time sequence.

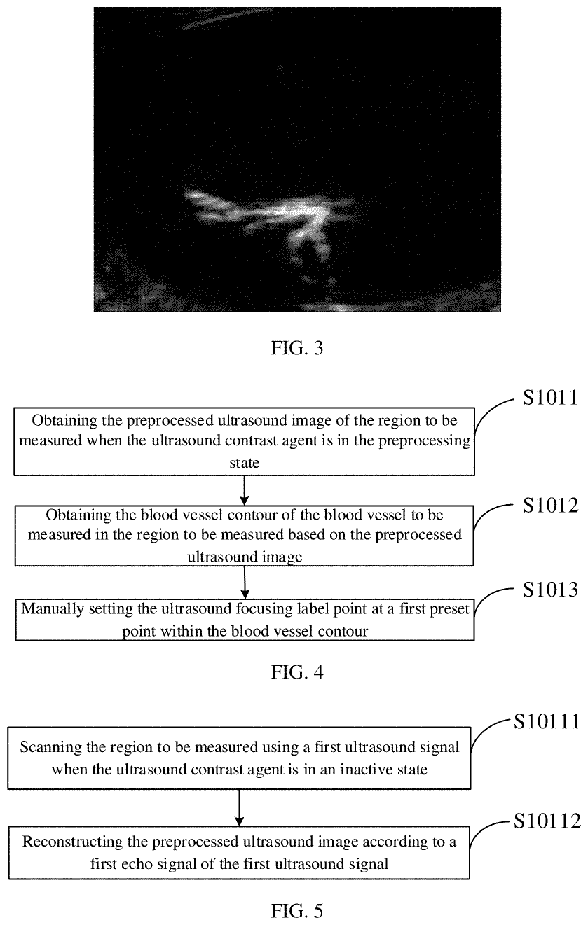

Since more and more nano-droplets in the ultrasound contrast agent are activated over time, the plurality of activation maps are arranged in time sequence, and the blood flow perfusion distribution map of the blood vessel to be measured can be obtained by processing the plurality of activation maps, as shown in FIG. 3 (FIG. 3 is the blood flow perfusion distribution image of the blood vessel to be measured according to the embodiment of this application).

In the embodiments of this application, the ultrasound focusing label point is set within the blood vessel contour of the blood vessel to be measured in the region to be measured. The ultrasound contrast agent is activated at the ultrasound focusing label point, so that the ultrasound contrast agent changes from the inactive state to the activated state, making the nano-droplets undergo the liquid-to-gas conversion from the nano-droplet state to the microbubble gas state, realizing the conversion of the ultrasound signal from dark to light, and making the ultrasound signal in the labeled blood vessel to be measured change from dark to light while other unlabeled blood vessel is still in the dark signal. The preactivated ultrasound image obtained under the dark signal is used as the background. Based on the activated ultrasound image obtained under the bright signal, the activation map of the ultrasound contrast agent in the blood vessel to be measured is obtained using the difference between the ultrasound images under the bright and the dark signal. The blood flow perfusion distribution map of the blood vessel to be measured is obtained based on the plurality of activation maps arranged in time sequence, so that the blood flow perfusion situation in the labeled blood vessel to be measured is more effectively highlighted.

FIG. 4 is a schematic flowchart of setting an ultrasound focusing label point in the ultrasonic blood perfusion imaging method for a single blood vessel according to an embodiment of this application. As shown in FIG. 4, setting the ultrasound focusing label point within a blood vessel contour of a blood vessel to be measured in a region to be measured may specifically include the following steps.

Step S1011: obtaining a preprocessed ultrasound image of the region to be measured when a ultrasound contrast agent is in a preprocessing state.

The ultrasound contrast agent that has a characteristic of liquid-to-gas conversion is injected. When the ultrasound contrast agent is in the preprocessing state, that is, when nano-droplets in the ultrasound contrast agent remain in liquid state, an ultrasound probe is used to detect the region to be imaged. When a contrast enhanced signal appears, the ultrasound probe starts to collect contrast data in order to obtain the preprocessed ultrasound image of the region to be measured.

The preprocessing state is different from the measuring state. In the preprocessing state, the nano-droplets in the ultrasound contrast agent are always in the nano-droplet state, and the nano-droplets will not be activated to cause liquid-to-gas conversion. In the measuring state, the ultrasound contrast agent will change from the inactive state to the activated state, and the nano-droplets will change from the liquid state to the gas state. In the measuring state, in order to obtain the blood flow perfusion situation, the ultrasound contrast agent needs to be activated from the inactive state to the activated state.

Step S1012: obtaining the blood vessel contour of the blood vessel to be measured in the region to be measured based on the preprocessed ultrasound image.

Due to the periodicity and randomness of the signal of liquid ultrasound contrast agents, the blood vessel contour of the blood vessel to be measured can be reflected in the preprocessed ultrasound image, and the blood vessel contour of the blood vessel to be measured within the region to be measured is determined manually.

Step S1013: manually setting the ultrasound focusing label point at a first preset point within the blood vessel contour.

The ultrasound focusing label point is a focal point that the ultrasound device focuses, which is used to activate the ultrasound contrast agent in the blood vessel to be measured, and the ultrasound focusing label point is manually set within the blood vessel contour for activation. Although the ultrasound focusing label point is a point manually set, its essence is a circular area with a diameter of 1 mm to 3 mm. In a preferred embodiment, the first preset point is set close to a source of the blood flow direction. Setting the first preset point at the source is beneficial to activating the ultrasound contrast agent.

In the embodiments of this application, the blood vessel contour to be measured is obtained through the preprocessed ultrasound image of the known region to be measured, and the ultrasound focusing label point is manually set in the blood vessel contour of the blood vessel to be measured, so as to provide a reference point to subsequently activate the ultrasound contrast agent in the blood vessel to be measured, thereby precisely activating the nano-droplets within the blood vessel to be measured.

FIG. 5 is a schematic flowchart of obtaining a preprocessed ultrasound image of the region to be measured in the ultrasonic blood perfusion imaging method for a single blood vessel according to the embodiment of this application. As shown in FIG. 5, obtaining the preprocessed ultrasound image of the region to be measured when the ultrasound contrast agent is in an inactive state may specifically include the following steps.

Step S10111: scanning the region to be measured using a first ultrasound signal when the ultrasound contrast agent is in an inactive state.

For example, the first ultrasound signal is a plane wave or a focused ultrasound sound beam, a pulse repetition frequency of the first ultrasound signal is 2-5 kHz, and a mechanical index of the first ultrasound signal is less than or equal to 0.8.

Step S10112: reconstructing the preprocessed ultrasound image according to a first echo signal of the first ultrasound signal.

In the embodiments of this application, by using the first ultrasound signal to scan the region to be measured, the preprocessed ultrasound image is reconstructed according to the first echo signal.

An executing device of steps S10111 and S10112 may be an array converter. As long as the above steps can be implemented, the embodiment of this application does not specifically limit the executing device.

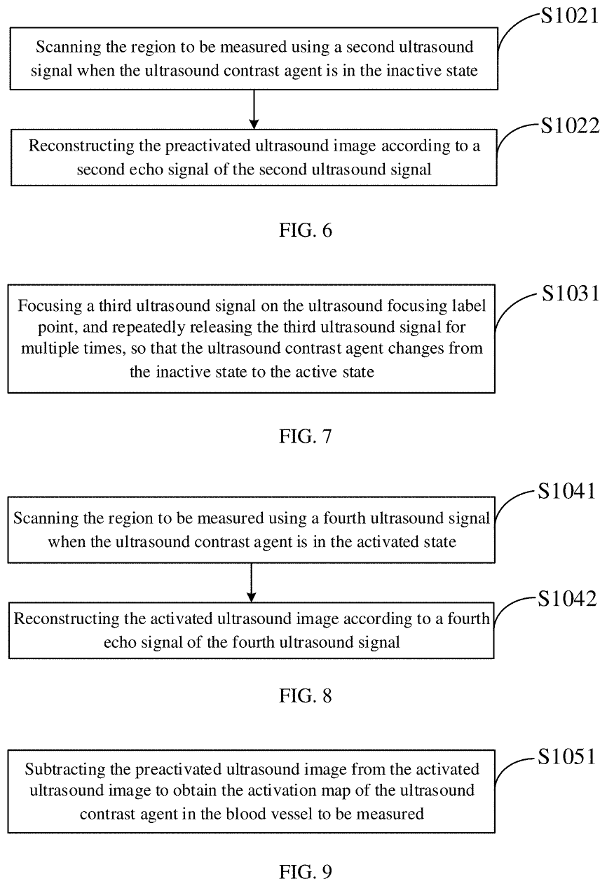

FIG. 6 is a schematic flowchart of obtaining a preactivated ultrasound image of a region to be measured in the ultrasonic blood perfusion imaging method for a single blood vessel according to the embodiment of this application. As shown in FIG. 6, obtaining the preactivated ultrasound image of the region to be measured when the ultrasound contrast agent is in an inactive state may specifically include the following steps.

Step S1021: scanning the region to be measured using a second ultrasound signal when the ultrasound contrast agent is in the inactive state.

For example, the second ultrasound signal is a plane wave, a pulse repetition frequency of the second ultrasound signal is 5 kHz, and a mechanical index of the second ultrasound signal is less than or equal to 0.8.

Step S1022: reconstructing the preactivated ultrasound image according to a second echo signal of the second ultrasound signal.

In the embodiments of this application, when the ultrasound contrast agent is in the inactive state, the region to be measured is scanned by using the second ultrasound signal. The preactivated ultrasound image is reconstructed according to the second echo signal. The preactivated ultrasound image of the region to be measured that is obtained under the dark signal serves as a subsequent background.

The executing device of step S1021 and step S1022 may be an array converter and may be the same array converter that performs step S10111 and step S10112. Different ultrasounds are emitted through the modulation of the array converter. As long as the above steps can be implemented, the embodiments of this application do not specifically limit the executing device.

FIG. 7 is a schematic flowchart of activating an ultrasound contrast agent in the ultrasonic blood perfusion imaging method for a single blood vessel according to the embodiment of this application. As shown in FIG. 7, activating the ultrasound contrast agent based on an ultrasound focusing label point may specifically include the following step.

Step S1031: focusing a third ultrasound signal on the ultrasound focusing label point, and repeatedly releasing the third ultrasound signal for multiple times, so that the ultrasound contrast agent changes from an inactive state to an activated state.

For example, the third ultrasound signal is a focused ultrasound wave, a pulse repetition frequency of the third ultrasound signal is 10 kHz, a mechanical index of the third ultrasound signal is 1.5-1.8, and an aperture of the third ultrasound signal is a full array element.

In the embodiments of this application, by focusing on the ultrasound focusing label point and repeatedly releasing the third ultrasound signal for multiple times, the ultrasound contrast agent is changed from the inactive state to the activated state, and nano-droplets in the blood vessel to be measured are changed from liquid to gas, which makes the ultrasound signal change from a dark signal to a bright signal. Therefore, the labeled blood vessel to be measured are in bright signal, while other unlabeled blood vessels are still in the dark signal, which more effectively highlights the distribution of nano-droplets in the ultrasound contrast agent in the labeled blood vessels to be measured, and then more effectively highlights the blood flow perfusion situation in the labeled blood vessel to be measured.

The executing device of step S1031 may be an array converter and may be the same array converter that performs step S10111 and step S10112 and performs step S1021 and step S1022. Different ultrasounds are emitted through the modulation of the array converter. As long as the above steps can be implemented, the embodiments of this application do not specifically limit the executing device.

In a further embodiment, the third ultrasound signal can be repeatedly released 3-5 times, so as to achieve the purpose of fully activating the nano-droplets in the ultrasound contrast agent near the ultrasound focusing label point. If the number of repeated releases of the third ultrasound signal is too small, the activation rate of the nano-droplets in the ultrasound contrast agent near the ultrasound focusing label point is affected, thereby affecting the display of blood flow perfusion situation. If the number of repeated releases of the third ultrasound signal is too great and the interval time is too long, the obtaining time of the blood flow perfusion distribution map is affected. Repeatedly releasing the third ultrasound signal for 3-5 times can not only achieve complete activation of the nano-droplets in the ultrasound contrast agent near the ultrasound focusing label point, but also ensure that the obtaining time of the blood flow perfusion distribution map is within a reasonable range.

FIG. 8 is a schematic flowchart of obtaining an activated ultrasound image of the region to be measured in the ultrasonic blood perfusion imaging method for a single blood vessel according to the embodiment of this application. As shown in FIG. 8, when the ultrasound contrast agent is in an activated state, obtaining the activated ultrasound image of the region to be measured may specifically include the following steps.

Step S1041: scanning the region to be measured using a fourth ultrasound signal when the ultrasound contrast agent is in the activated state.

For example, the fourth ultrasound signal is a plane wave, a pulse repetition frequency of the fourth ultrasound signal is 5 kHz, and a mechanical index of the fourth ultrasound signal is less than or equal to 0.8.

Step S1042: reconstructing the activated ultrasound image according to a fourth echo signal of the fourth ultrasound signal.

In the embodiments of this application, when the ultrasound contrast agent is in the activated state, the region to be measured is scanned by using the fourth ultrasound signal, and according to the fourth echo signal, the activated ultrasound image with the labeled blood vessel to be measured being in the bright signal and other unlabeled blood vessels being still in the dark signal is reconstructed. Based on the activated ultrasound image of the region to be measured obtained under the bright signal, a difference between the labeled blood vessel to be measured and other surrounding blood vessels is reflected in real time.

The executing device of step S1041 and step S1042 may be an array converter, and may be the same array converter that performs step S10111 and step S10112, performs step S1021 and step S1022, and performs step S1031. Different ultrasounds are emitted through the modulation of the array converter. As long as the above steps can be implemented, the embodiment of this application does not specifically limit the executing device.

FIG. 9 is a schematic flowchart of obtaining an activation map of the ultrasound contrast agent in a blood vessel to be measured in the ultrasonic blood perfusion imaging method for a single blood vessel according to the embodiment of this application. As shown in FIG. 9, obtaining the activation map of the ultrasound contrast agent in the blood vessel to be measured based on a preactivated ultrasound image and an activated ultrasound image may specifically include the following step.

Step S1051: subtracting the preactivated ultrasound image from the activated ultrasound image to obtain the activation map of the ultrasound contrast agent in the blood vessel to be measured.

For example, the activated ultrasound image and the preactivated ultrasound image have a same resolution and size.

The preactivated ultrasound image is an ultrasound image when the ultrasound contrast agent is in an inactive state and is an ultrasound image when the blood vessel to be measured and its surroundings are all under the dark signal. The activated ultrasound image is the ultrasound image when the ultrasound contrast agent is in the activated state and is the ultrasound image when the blood vessel to be measured is under the bright signal and its surroundings are all under the dark signal. The preactivated ultrasound image is subtracted from the activated ultrasound image to subtract the background image that has not changed. The activation map of the ultrasound contrast agent in the blood vessel to be measured is obtained, that is, the activation map of the nano-droplets in the blood vessel to be measured is obtained.

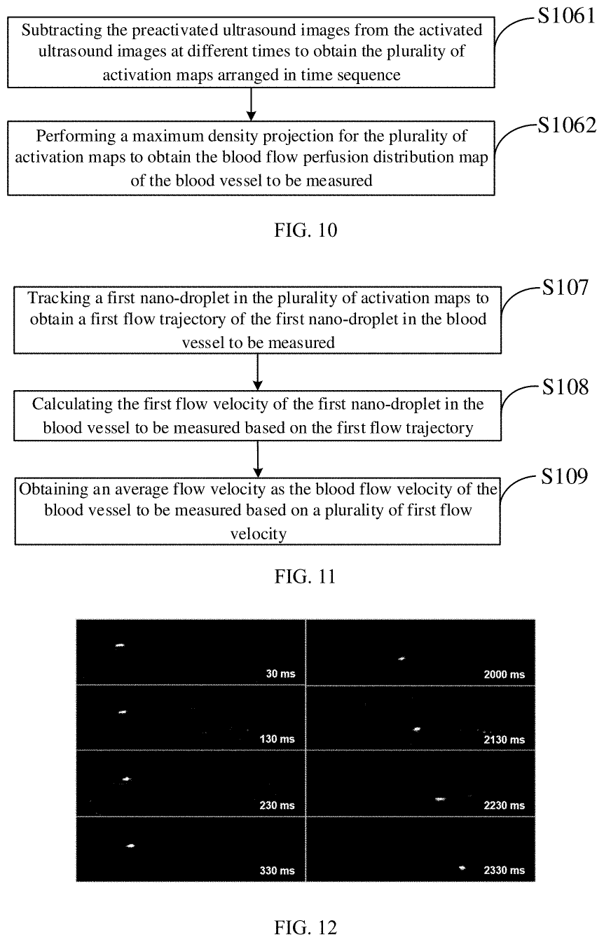

FIG. 10 is a schematic flowchart of obtaining a blood flow perfusion distribution map of the blood vessel to be measured based on a plurality of activation maps arranged in time sequence in the ultrasonic blood perfusion imaging method for a single blood vessel according to the embodiment of this application. As shown in FIG. 10, obtaining a blood flow perfusion distribution map of the blood vessel to be measured based on a plurality of activation maps arranged in time sequence may specifically include the following steps.

Step S1061: subtracting a preactivated ultrasound images from an activated ultrasound images at different times to obtain a plurality of activation maps arranged in time sequence.

Since the fluidity of the blood itself will drive the flow of the ultrasound contrast agent, the activated ultrasound contrast agent has different displayed positions in the activated ultrasound images at different times. By subtracting the preactivated ultrasound images from the activated ultrasound images at different times, the plurality activation maps arranged in time sequence can reflect the flow of the activated ultrasound contrast agent in the blood vessel to be measured.

Step S1062: performing a maximum density projection for the plurality of activation maps to obtain the blood flow perfusion distribution map of the blood vessel to be measured.

Based on the plurality of activation maps arranged in time sequence, the maximum density projection can be performed to obtain the blood flow perfusion distribution map of the blood vessel to be measured.

In the embodiment of this application, by subtracting the preactivated ultrasound images from the activated ultrasound images at different times, a plurality of activation maps arranged in time sequence that can reflect the flow of the activated ultrasound contrast agent in the blood vessel to be measured are obtained, and the blood flow perfusion distribution map of the blood vessel to be measured is obtained by performing maximum density projection for the plurality of activation maps (as shown in FIG. 3).

FIG. 11 is a schematic flowchart of an ultrasonic blood perfusion imaging method for a single blood vessel according to the embodiment of this application. As shown in FIG. 11, after obtaining activation maps of the ultrasound contrast agent in the blood vessel to be measured, the ultrasonic blood perfusion imaging method for a single blood vessel further includes the following steps.

Step S107: tracking a first nano-droplet in the plurality of activation maps to obtain a first flow trajectory of the first nano-droplet in the blood vessel to be measured.

FIG. 12 is a schematic diagram showing the positions of a first nano-droplet in the activated ultrasound images at different times according to the embodiment of this application. As shown in FIG. 12, the positions of the first nano-droplet in the activated ultrasound images at different times are different, because the nano-droplets in the ultrasound contrast agent also flow with the blood flow. Due to the flow, the first nano-droplet activated at the ultrasound focusing label point is at different positions in the activated ultrasound images obtained at different times. By connecting the positions of the first nano-droplet in the activated ultrasound images at different times, a first flow trajectory of the first nano-droplet in the blood vessel to be measured can be obtained.

The first in the first nano-droplet is just a pronoun, which can be any nano-droplet in the ultrasound contrast agent in the blood vessel to be measured, as long as a same nano-droplet is tracked.

Step S108: calculating a first flow velocity of the first nano-droplet in the blood vessel to be measured based on the first flow trajectory.

The first flow velocity can be obtained by obtaining the displacement per unit time according to the first flow trajectory.

Step S109: obtaining an average flow velocity as the blood flow velocity of the blood vessel to be measured based on a plurality of first flow velocity.

Since the ultrasound contrast agent includes a plurality of first nano-droplets, and each first nano-droplet corresponds to a first flow velocity, the average flow velocity is obtained according to the plurality of first flow velocity, and the average flow velocity is obtained as the blood flow velocity of the blood vessel to be measured, reflecting the blood flow velocity of the blood vessel to be measured.

In the embodiments of this application, the first flow trajectory is obtained by tracking the first nano-droplet in the activated ultrasound images at different times, the first flow velocity is obtained according to the first flow trajectory, and the average flow velocity is obtained according to a plurality of first flow velocity, reflecting the blood flow velocity of the blood vessel to be measured

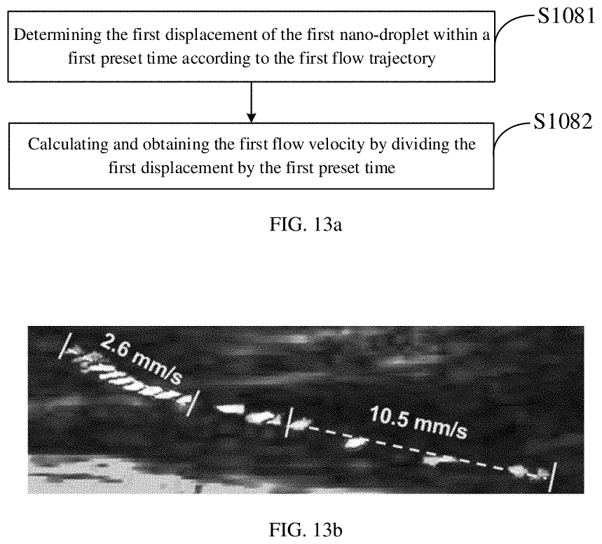

FIG. 13a is a schematic flowchart of calculating a first flow velocity of a first nano-droplet in the blood vessel to be measured in the ultrasonic blood perfusion imaging method for a single blood vessel according to the embodiment of this application. As shown in FIG. 13a, calculating the first flow velocity of the first nano-droplet in the blood vessel to be measured based on a first flow trajectory may specifically include the following steps.

Step S1081: determining a first displacement of the first nano-droplet within a first preset time according to the first flow trajectory.

As shown in FIG. 12, the plurality of activated ultrasound images of the first nano-droplet at different times are compared, and the first displacement within a first preset time is obtained. For example, as shown in FIG. 12, a distance 51 between the first nano-droplet in the activated ultrasound image after 30 ms and the first nano-droplet in the activated ultrasound image after 2330 ms is measured to obtain the first displacement.

Step S1082: calculating and obtaining the first flow velocity by dividing the first displacement by the first preset time.

The first flow velocity is obtained by dividing the first displacement by the first preset time, for example: dividing the distance 51 between the first nano-droplet in the activated ultrasound image after 30 ms and the first nano-droplet in the activated ultrasound image after 2330 ms by 2300 ms to obtain the first flow velocity, as shown in FIG. 13b (FIG. 13b is a blood flow velocity diagram in the blood vessel to be measured according to the embodiment of this application).

In the embodiments of this application, the plurality of activated ultrasound images of the first nano-droplet at different times are compared, the first displacement within the first preset time is obtained, and the first flow velocity is obtained by dividing the first displacement by the first preset time. The flow velocity of the nano-droplets in the ultrasound contrast agent can be reflected more accurately, so as to accurately reflect the blood flow velocity in the blood vessel to be measured.

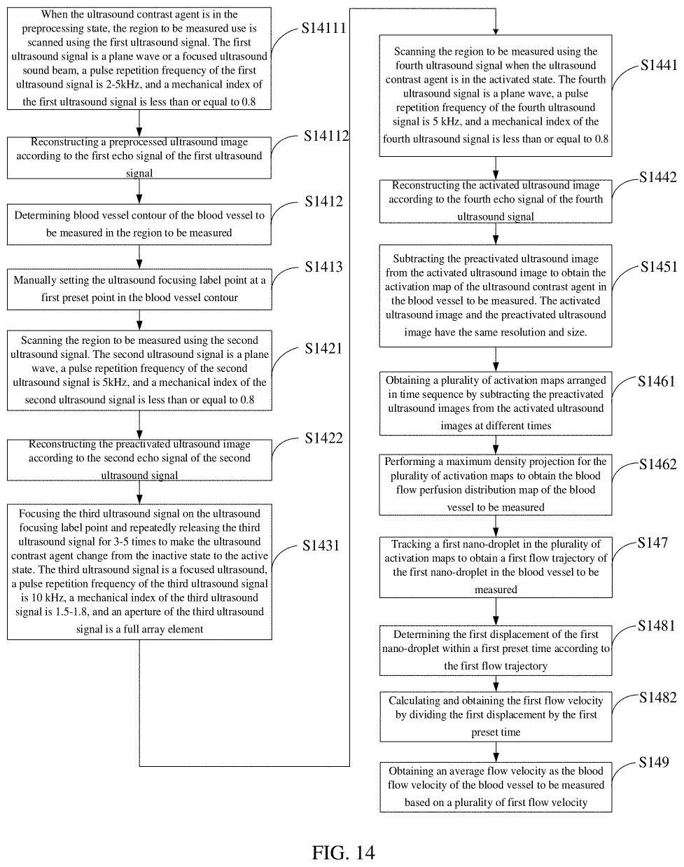

FIG. 14 is a schematic flowchart of the ultrasonic blood perfusion imaging method for a single blood vessel according to the embodiment of this application.

A pre-imaging mode is the mode in which a preprocessed ultrasound image is obtained when the ultrasound contrast agent is in a preprocessing state, and the measurement mode is the mode in which the ultrasound contrast agent is changed from an inactive state to an activated state, thereby measuring the blood flow perfusion situation of the labeled single blood vessel. In the pre-imaging mode, when the ultrasound contrast agent is in the preprocessing state, the region to be measured is scanned using a first ultrasound signal. The first ultrasound signal is a plane wave or a focused ultrasound sound beam, a pulse repetition frequency of the first ultrasound signal is 2-5 kHz, and a mechanical index of the first ultrasound signal is less than or equal to 0.8 (as shown in step S14111 in FIG. 14). According to a first echo signal of the first ultrasound signal, a preprocessed ultrasound image is reconstructed (as shown in step S14112 in FIG. 14). According to the periodicity and randomness of the signal due to the liquid ultrasound contrast agent, a blood vessel contour of the blood vessel to be measured can be reflected in the preprocessed ultrasound image, the blood vessel contour of the blood vessel to be measured in the region to be measured can be displayed in the preprocessed ultrasound image, and the blood vessel contour of the blood vessel to be measured in the region to be measured is manually determined (as shown in step S1412 in FIG. 14). An ultrasound focusing label point is manually set at a first preset point in the blood vessel contour. By manually setting the ultrasound focusing label point in the blood vessel contour of the blood vessel to be measured, the reference point for the subsequent activation of the ultrasound contrast agent in the blood vessel to be measured is provided (as shown in step S1413 in FIG. 14).

In the measurement mode, the region to be measured is scanned using a second ultrasound signal. The second ultrasound signal is a plane wave, a pulse repetition frequency of the second ultrasound signal is 5 kHz, and a mechanical index of the second ultrasound signal is less than or equal to 0.8 (as shown in step S1421 in FIG. 14). A preactivated ultrasound image is reconstructed according to a second echo signal of the second ultrasound signal (as shown in step S1422 in FIG. 14). A third ultrasound signal is focused on the ultrasound focusing label point, and the third ultrasound signal is repeatedly released for 3-5 times. The third ultrasound signal is a focused ultrasound wave, a pulse repetition frequency of the third ultrasound signal is 10 kHz, a mechanical index of the third ultrasound signal is 1.5-1.8, and an aperture of the third ultrasound signal is a full array element. The ultrasound contrast agent changes from an inactive state to an activated state, the nano-droplets in the blood vessel to be measured change from the liquid state to the gas state, and the ultrasound signal changes from the dark signal to the bright signal, so that the labeled blood vessel to be measured is in the bright signal and other unlabeled blood vessels are still in a dark signal (as shown in step S1431 in FIG. 14). The region to be measured is scanned using a fourth ultrasound signal, the fourth ultrasound signal is a plane wave, a pulse repetition frequency of the fourth ultrasound signal is 5 kHz, and a mechanical index of the fourth ultrasound signal is less than or equal to 0.8 (as shown in step S1441 in FIG. 14). An activated ultrasound image is reconstructed according to a fourth echo signal (as shown in step S1442 in FIG. 14). The activated ultrasound image with the labeled blood vessel to be measured being in the bright signal and other unlabeled blood vessels being still in dark signal is reconstructed. Based on the activated ultrasound image of the region to be measured obtained under the bright signal, the difference between the labeled blood vessels to be measured and other surrounding vessels are reflected in real time.

The preactivated ultrasound image is the ultrasound image when the ultrasound contrast agent is in the inactive state and is the ultrasound image when the blood vessel to be measured and its surroundings are all dark signals. The activated ultrasound image is the ultrasound image when the ultrasound contrast agent is in the activated state and is the ultrasound image when the blood vessel to be measured is under the bright signal and other unlabeled blood vessels are all under the dark signal. The preactivated ultrasound image is subtracted from the activated ultrasound image to subtract the background image that has not changed. An activation map of the ultrasound contrast agent in the blood vessel to be measured is obtained, that is, the activation map of the nano-droplets in the blood vessel to be measured is obtained (as shown in step S1451 in FIG. 14). By subtracting the preactivated ultrasound images from the activated ultrasound images at different times, a plurality of activation maps arranged in time sequence can be obtained (as shown in step S1461 in FIG. 14), reflecting the flow situation of the activated ultrasound contrast agent in the blood vessel to be measured. A maximum density projection for the plurality of activation maps is performed to obtain the blood flow perfusion distribution map of the blood vessel to be measured (as shown in step S1462 in FIG. 14).

The positions of the first nano-droplet activated at the ultrasound focusing label point are different in the activated ultrasound images obtained at different times due to the flow. By connecting the positions of the first nano-droplet in the activated ultrasound images at different times, a first flow trajectory of the first nano-droplet in the blood vessel to be measured can be obtained (as shown in step S147 in FIG. 14). The plurality of activated ultrasound images of the first nano-droplet at different times are compared to obtain a first displacement within the first preset time (as shown in step S1481 in FIG. 14). A first flow velocity is obtained by dividing the first displacement by the first preset time (as shown in step S1482 in FIG. 14). Each first nano-droplet corresponds to one first flow velocity, and according to the average of the plurality of first flow velocity, an average flow velocity is obtained as the blood flow velocity of the blood vessel to be measured, reflecting the blood flow velocity of the blood vessel to be measured, and the average flow velocity is obtained as the blood flow velocity of the blood vessel to be measured (as shown in step S149 in FIG. 14).

Through the above method, a blood flow perfusion distribution map of the blood vessel to be measured can be obtained, the blood flow velocity of the blood vessel to be measured can be obtained, the blood flow perfusion situation of a specific blood vessel can be accurately observed, and the blood flow perfusion characteristic of a single blood vessel can be observed in real time under ultrasound.

Exemplary Apparatus for Single Blood Vessel Ultrasound Blood Flow Perfusion Imaging

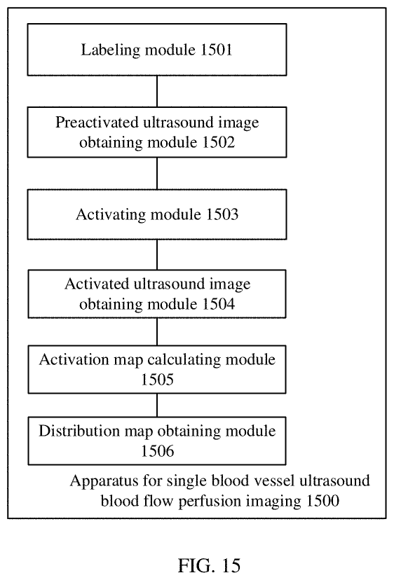

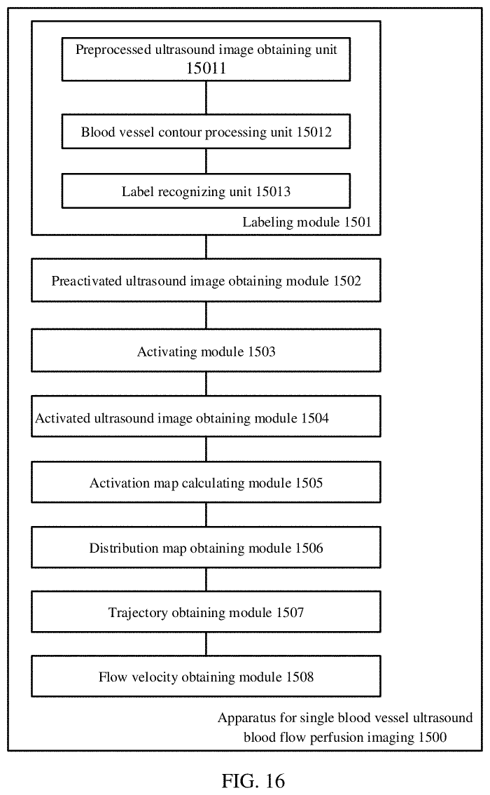

FIG. 15 is a schematic diagram of an ultrasonic blood perfusion imaging apparatus for a single blood vessel according to an embodiment of this application. As shown in FIG. 15, the ultrasonic blood perfusion imaging apparatus for a single blood vessel 1500 includes: a labeling module 1501, configured to set an ultrasound focusing label point within a blood vessel contour of a blood vessel to be measured in a region to be measured; a preactivated ultrasound image obtaining module 1502, configured to obtain a preactivated ultrasound image of the region to be measured when an ultrasound contrast agent is in an inactive state, the ultrasound contrast agent including a plurality of nano-droplets, and the nano-droplets being in a nano-droplet state when the ultrasound contrast agent is in an inactive state; an activating module 1503, configured to activate the ultrasound contrast agent based on the ultrasound focusing label point; an activated ultrasound image obtaining module 1504, configured to obtain an activated ultrasound image of the region to be measured when the ultrasound contrast agent is in an activated state, the nano-droplets are in a microbubble gas state when the ultrasound contrast agent is in an activated state; an activation map calculating module 1505, configured to obtain an activation map of the ultrasound contrast agent in the blood vessel to be measured based on the preactivated ultrasound image and the activated ultrasound image; and a distribution map obtaining module 1506, configured to obtain the blood flow perfusion distribution map of the blood vessel to be measured based on a plurality of activation maps arranged in time sequence.

In the embodiment of this application, the labeling module 1501 is used to set the ultrasound focusing label point in the blood vessel contour of the blood vessel to be measured in the region to be measured; the activating module 1503 is used to activate the ultrasound contrast agent at the ultrasound focusing label point so that the ultrasound contrast agent changes from the activated state to the activated state, making the nano-droplets undergo the liquid-to-gas conversion from the nano-droplet state to the microbubble gas state, realizing the conversion of the ultrasound signal from dark to light, and making the ultrasound signal in the labeled blood vessel to be measured from dark to light while other unlabeled blood vessel is still in the dark signal. The preactivated ultrasound image obtained by the preactivated ultrasound image obtaining module 1502 under the dark signal is used as the background. Based on the activated ultrasound image obtained by the activated post-activation ultrasound image obtaining module 1504 under the bright signal, the activation map of the ultrasound contrast agent in the blood vessel to be measured is obtained by the activation map calculating module 1505 using the difference between the ultrasound images under the bright and the dark signal. The blood flow perfusion distribution map of the blood vessel to be measured is obtained by the distribution map obtaining module 1506 based on the plurality of activation maps arranged in time sequence, so that the blood flow perfusion situation in the labeled blood vessel to be measured is more effectively highlighted.

In a further embodiment, the preactivated ultrasound image obtaining module 1502, the activating module 1503, and the activated ultrasound image obtaining module 1504 may be executed by an array converter, and the array converter executes the steps performed by the above three modules to achieve functions of obtaining the preactivated ultrasound image, activating the ultrasound contrast agent, and obtaining the activated ultrasound image.

FIG. 16 is a schematic diagram of an ultrasonic blood perfusion imaging apparatus for a single blood vessel according to an embodiment of this application. As shown in FIG. 16, the labeling module 1501 specifically includes: a preprocessed ultrasound image obtaining unit 15011, configured to obtain a preprocessed ultrasound image of the region to be measured when the ultrasound contrast agent is in a preprocessing state; a blood vessel contour processing unit 15012, configured to obtain the blood vessel contour of the blood vessel to be measured in the region to be measured based on the preprocessed ultrasound image, and send it to the display operation interface; a label recognizing unit 15013, configured to identify the manually set ultrasound focusing label point for subsequent activation of the ultrasound contrast agent.

In a further embodiment, the preprocessed ultrasound image obtaining unit 15011 may be performed by an array converter.

In a further embodiment, in the case that the ultrasonic blood perfusion imaging apparatus for a single blood vessel is manually adjusted to enter a pre-imaging mode, and when the ultrasound contrast agent is in the preprocessing state, the region to be measured use is scanned by the array converter using the first ultrasound signal. The first ultrasound signal is a plane wave or a focused ultrasound sound beam, a pulse repetition frequency of the first ultrasound signal is 2-5 kHz, and a mechanical index of the first ultrasound signal is less than or equal to 0.8. According to the first echo signal of the first ultrasound signal, a preprocessed ultrasound image is reconstructed. In the case that the ultrasonic blood perfusion imaging apparatus for a single blood vessel is manually adjusted to enter the measurement mode, and when the ultrasound contrast agent is in an inactive state, the region to be measured is scanned by the array converter using the second ultrasound signal. The second ultrasound signal is a plane wave, a pulse repetition frequency of the second ultrasound signal is 5 kHz, and a mechanical index of the second ultrasound signal is less than or equal to 0.8. The blood vessel contour processing unit 15012 is used to obtain the blood vessel contour of the blood vessel to be measured based on the preprocessed ultrasound image and send it to the display operation interface. The label recognizing unit 15013 is used to identify the manually set ultrasound focusing label point. The third ultrasound signal is focused on the ultrasound focusing label point, and the third ultrasound signal is repeatedly released by the array converter for 3-5 times to make the ultrasound contrast agent changing from the inactive state to the activated state. The third ultrasound signal is a focused ultrasound wave, a pulse repetition frequency of the third ultrasound signal is 10 kHz, a mechanical index of the third ultrasound signal is 1.5-1.8, and an aperture of the third ultrasound signal is a full array element. After the ultrasound contrast agent is activated by the third ultrasound signal, when the ultrasound contrast agent is in the activated state, the region to be measured is scanned by the fourth ultrasound signal emitted by the array converter, the fourth ultrasound signal is a plane wave, a pulse repetition frequency of the fourth ultrasound signal is 5 kHz, and a mechanical index of the fourth ultrasound signal is less than or equal to 0.8. The activated ultrasound image is reconstructed according to the fourth echo signal.

In one embodiment, the activation map calculating module 1505 is further configured to subtract the preactivated ultrasound image from the activated ultrasound image to obtain the activation map of the ultrasound contrast agent in the blood vessel to be measured. The activated ultrasound image and the preactivated ultrasound image have a same resolution and size.

In one embodiment, the activation map calculating module 1505 is further configured to subtract the preactivated ultrasound images from the activated ultrasound images at different times to obtain a plurality of activation maps arranged in time sequence. The distribution map obtaining module 1506 further includes: a maximum density projecting unit configured to perform a maximum density projection for the plurality of activation maps to obtain the blood flow perfusion distribution map of the blood vessel to be measured.

In one embodiment, the ultrasonic blood perfusion imaging apparatus for a single blood vessel 1500 further includes: a trajectory obtaining module 1507, configured to track a first nano-droplet in the plurality of activation maps and obtain a first flow trajectory of the first nano-droplet in the blood vessel to be measured; and a flow velocity obtaining module 1508, configured to calculate a first flow velocity of the first nano-droplet in the blood vessel to be measured based on the first flow trajectory and obtain an average flow velocity as the blood flow velocity of the blood vessel to be measured based on the plurality of first flow velocity.

In one embodiment, the flow velocity obtaining module 1508 is further configured to determine a first displacement of the first nano-droplet within a first preset time according to the first flow trajectory and calculate and obtain the first flow velocity by dividing the first displacement by the first preset time.

For the specific functions and operations of each module and unit in the above ultrasonic blood perfusion imaging apparatus for a single blood vessel, reference may be made to the ultrasonic blood perfusion imaging method for a single blood vessel according to the above-mentioned FIGS. 1, 4 to 11, FIG. 13a, and FIG. 14. In order to avoid repetition, details are not repeated here.

Exemplary Electronics

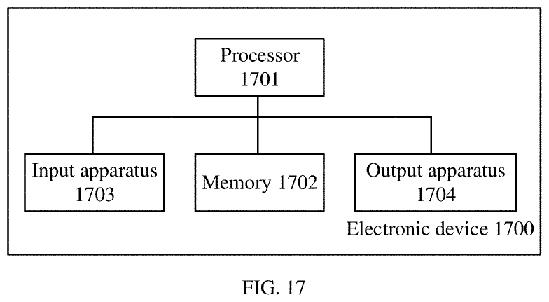

FIG. 17 is a schematic structural diagram of an electronic device according to an embodiment of this application. As shown in FIG. 17, the electronic device 1700 includes:

one or more processors 1701 and a memory 1702; and computer program instructions stored in the memory 1702, the computer program instructions, when executed by the processor 1701, causing the processor 1701 to execute the ultrasonic blood perfusion imaging method for a single blood vessel according to any one of the above embodiments.

The processor 1701 may be a central processing unit (CPU) or other form of processing unit with data processing capabilities and/or instruction executing capabilities and can control other components in the electronic device to perform desired functions.

Memory 1702 may include one or more computer program products, which may include various forms of computer-readable storage medium, such as volatile memory and/or non-volatile memory. The volatile memory may include, for example, random access memory (RAM) and/or cache memory (cache). The non-volatile memory may include, for example, a read only memory (ROM), a hard disk, a flash memory, and the like. One or more computer program instructions may be stored on the computer-readable storage medium, and the processor 1701 may execute the program instructions to implement steps in the ultrasonic blood perfusion imaging method for a single blood vessel in any of the above-mentioned embodiments and/or other desired functions. Information such as the pre-imaging mode, the measurement mode, and manually identified points may also be stored in the computer-readable storage medium.

In one example, the electronic device 1700 may also include an input apparatus 1703 and an output apparatus 1704 interconnected by a bus system and/or other form of connection mechanism (not shown in FIG. 17).

In addition, the input apparatus 1703 may also include, for example, a keyboard, a mouse, a microphone, and the like.

The output apparatus 1704 can output various information to the outside, for example, it can include, for example, a display, a speaker, a printer, a communication network and its connected remote output device, and the like.

Of course, for simplicity, only some of the components in the electronic device 1700 related to this application are shown in FIG. 17, and components such as a bus, an input apparatus/output interface and the like are omitted. Besides, the electronic device 1700 may also include any other suitable components according to the specific application.

Exemplary computer program product and computer readable storage medium

In addition to the above methods and apparatuses, the embodiments of this application may also be computer program products, including computer program instructions that, when executed by a processor, cause the processor to perform the steps in the ultrasonic blood perfusion imaging method for a single blood vessel as described in any of the foregoing embodiments.

The computer program product can write program code for performing the operations of the embodiments of this application in any combination of one or more programming languages, including object-oriented programming languages, such as Java, C++, etc., and also includes conventional procedural programming languages, such as the “C” language or similar programming languages. The program code may be executed entirely on the user's computing device, partly on the user's device, as a stand-alone software package, partly on the user's computing device and partly on a remote computing device, or entirely on the remote computing device or server.

In addition, embodiments of this application may also be computer-readable storage medium having computer program instructions stored thereon, the computer program instructions, when executed by a processor, cause the processor to perform the steps of the ultrasonic blood perfusion imaging method for a single blood vessel according to any of the above-mentioned embodiments of this application are described in the “Exemplary Method for Single blood vessel Ultrasound Perfusion Imaging” section.

The computer-readable storage medium may employ any combination of one or more readable medium. The readable medium may be a readable signal medium or a readable storage medium. The readable storage medium may include, for example, but not limited to, electrical, magnetic, optical, electromagnetic, infrared, or semiconductor systems, apparatuses or equipment, or a combination of any of the above. More specific examples (a non-exhaustive list) of readable storage medium include: an electrical connection having one or more wires, a portable disk, a hard disk, a random access memory (RAM), a read only memory (ROM), an erasable programmable read only memory (EPROM or flash memory), a fiber optic, a portable compact disk read only memory (CD-ROM), an optical storage device, a magnetic storage device, or any suitable combination of the above.

The basic principles of this application have been described above in conjunction with specific embodiments. However, it should be pointed out that the advantages, advantages, effects, etc. mentioned in this application are only examples rather than limitations, and these advantages, advantages, effects, etc., are not considered to be required for each embodiment of this application. In addition, the specific details disclosed above are only for the purpose of example and easy understanding, rather than limiting, and the above-mentioned details do not limit the application to be implemented by using the above-mentioned specific details.

The block diagrams of equipment, apparatus, devices, and systems referred to in this application are merely illustrative examples and are not intended to require or imply that the connections, arrangements, or configurations must be in the manner shown in the block diagrams. As those skilled in the art will appreciate, these equipment, apparatus, devices, and systems may be connected, arranged, configured in any manner. Words such as “including”, “including”, “having” and the like are open-ended words meaning “including but not limited to” and are used interchangeably therewith. As used herein, the words “or” and “and” refer to and are used interchangeably with the word “and/or” unless the context clearly dictates otherwise. As used herein, the word “such as” refers to and is used interchangeably with the phrase “such as but not limited to”.

It should also be pointed out that in the apparatus, device and method of this application, each component or each step can be decomposed and/or recombined. These disaggregations and/or recombinations should be considered as equivalents of this application.

The above description of the disclosed aspects is provided to enable any person skilled in the art to make or use this application. Various modifications to these aspects will be readily apparent to those skilled in the art, and the generic principles defined herein may be applied to other aspects without departing from the scope of the application. Therefore, this application is not intended to be limited to the aspects shown herein but is to be accorded the widest scope consistent with the principles and novel features disclosed herein.

The foregoing description has been presented for the purposes of illustration and description. Furthermore, this description is not intended to limit the embodiments of the application to the forms disclosed herein. Although a number of example aspects and embodiments have been discussed above, those skilled in the art will recognize certain variations, modifications, changes, additions and sub-combinations thereof.

What is claimed is:

1. An ultrasonic blood perfusion imaging method for a single blood vessel, comprising:
setting an ultrasound focusing label point within a blood vessel contour of a blood vessel to be measured in a region to be measured;
obtaining a preactivated ultrasound image of the region to be measured when an ultrasound contrast agent is in an inactive state, the ultrasound contrast agent comprising a plurality of nano-droplets, and the nano-droplets being in a nano-droplet state when the ultrasound contrast agent is in the inactive state;
activating the ultrasound contrast agent based on the ultrasound focusing label point;
obtaining an activated ultrasound image of the region to be measured when the ultrasound contrast agent is in an activated state;
obtaining an activation map of the ultrasound contrast agent in the blood vessel to be measured based on the preactivated ultrasound image and the activated ultrasound image, the nano-droplets being in a microbubble gas state when the ultrasound contrast agent is in an activated state; and
obtaining a blood flow perfusion distribution map of the blood vessel to be measured based on a plurality of activation maps arranged in time sequence.

2. The ultrasonic blood perfusion imaging method for a single blood vessel according to claim 1, wherein the setting an ultrasound focusing label point within a blood vessel contour of a blood vessel to be measured in a region to be measured comprises:
obtaining a preprocessed ultrasound image of the region to be measured when the ultrasound contrast agent is in a preprocessing state;
obtaining the blood vessel contour of the blood vessel to be measured in the region to be measured based on the preprocessed ultrasound image; and
manually setting the ultrasound focusing label point at a first preset point within the blood vessel contour.

3. The ultrasonic blood perfusion imaging method for a single blood vessel according to claim 2, wherein the obtaining a preprocessed ultrasound image of the region to be measured when an ultrasound contrast agent is in a preprocessing state comprises:
scanning the region to be measured using a first ultrasound signal when the ultrasound contrast agent is in the preprocessing state; and
reconstructing the preprocessed ultrasound image according to a first echo signal of the first ultrasound signal.

4. The ultrasonic blood perfusion imaging method for a single blood vessel according to claim 3, wherein the first ultrasound signal is a plane wave or a focused ultrasound sound beam, a pulse repetition frequency of the first ultrasound signal is 2-5 kHz, and a mechanical index of the first ultrasound signal is less than or equal to 0.8.

5. The ultrasonic blood perfusion imaging method for a single blood vessel according to claim 1, wherein the obtaining a preactivated ultrasound image of the region to be measured when an ultrasound contrast agent is in an inactive state comprises:
scanning the region to be measured using a second ultrasound signal when the ultrasound contrast agent is in the inactive state; and
reconstructing the preactivated ultrasound image according to a second echo signal of the second ultrasound signal.

6. The ultrasonic blood perfusion imaging method for a single blood vessel according to claim 5, wherein the second ultrasound signal is a plane wave, a pulse repetition frequency of the second ultrasound signal is 5 kHz, and a mechanical index of the second ultrasound signal is less than or equal to 0.8.

7. The ultrasonic blood perfusion imaging method for a single blood vessel according to claim 5, wherein the activating the ultrasound contrast agent based on the ultrasound focusing label point comprises:
focusing a third ultrasound signal on the ultrasound focusing label point, and repeatedly releasing the third ultrasound signal for multiple times, so that the ultrasound contrast agent changes from the inactive state to the active state.

8. The ultrasonic blood perfusion imaging method for a single blood vessel according to claim 7, wherein the third ultrasound signal is a focused ultrasound wave, a pulse repetition frequency of the third ultrasound signal is 10 kHz, a mechanical index of the third ultrasound signal is 1.5-1.8, and the aperture of the third ultrasound signal is a full array element.

9. The ultrasonic blood perfusion imaging method for a single blood vessel according to claim 7, wherein a number of times of releasing the third ultrasound signal is 3-5.

10. The ultrasonic blood perfusion imaging method for a single blood vessel according to claim 7, wherein the obtaining an activated ultrasound image of the region to be measured when the ultrasound contrast agent is in an activated state comprises:
scanning the region to be measured using a fourth ultrasound signal when the ultrasound contrast agent is in the activated state; and
reconstructing the activated ultrasound image according to a fourth echo signal of the fourth ultrasound signal.

11. The ultrasonic blood perfusion imaging method for a single blood vessel according to claim 10, wherein the fourth ultrasound signal is a plane wave, a pulse repetition frequency of the fourth ultrasound signal is 5 kHz, and a mechanical index of the fourth ultrasound signal is less than or equal to 0.8.

12. The ultrasonic blood perfusion imaging method for a single blood vessel according claim 1, wherein the obtaining an activation map of the ultrasound contrast agent in the blood vessel to be measured based on the preactivated ultrasound image and the activated ultrasound image comprises:
subtracting the preactivated ultrasound image from the activated ultrasound image to obtain the activation map of the ultrasound contrast agent in the blood vessel to be measured;
wherein the activated ultrasound image and the preactivated ultrasound image have a same resolution and size.

13. The ultrasonic blood perfusion imaging method for a single blood vessel according to claim 1, wherein the obtaining a blood flow perfusion distribution map of the blood vessel to be measured based on a plurality of activation maps arranged in time sequence comprises:
subtracting the preactivated ultrasound images from the activated ultrasound images at different times to obtain a plurality of activation maps arranged in time sequence; and
performing a maximum density projection for the plurality of activation maps to obtain the blood flow perfusion distribution map of the blood vessel to be measured.

14. The ultrasonic blood perfusion imaging method for a single blood vessel according to claim 1, wherein after the obtaining an activation map of the ultrasound contrast agent in the blood vessel to be measured, the method further comprises:
tracking a first nano-droplet in a plurality of the activation maps to obtain a first flow trajectory of the first nano-droplet in the blood vessel to be measured;
calculating a first flow velocity of the first nano-droplet in the blood vessel to be measured based on the first flow trajectory; and
obtaining an average flow velocity as a blood flow velocity of the blood vessel to be measured based on a plurality of the first flow velocity.

15. The ultrasonic blood perfusion imaging method for a single blood vessel according to claim 14, wherein the calculating a first flow velocity of the first nano-droplet in the blood vessel to be measured based on the first flow trajectory comprises:
determining a first displacement of the first nano-droplet within a first preset time according to the first flow trajectory; and
calculating and obtaining the first flow velocity by dividing the first displacement by the first preset time.

16. An electronic device, comprising:
a processor; and
a memory for storing computer program instructions, the computer program instructions, when executed by the processor, causing the processor to perform the ultrasonic blood perfusion imaging method for a single blood vessel according to claim 1.

17. A non-transitory computer-readable storage medium, wherein computer program instructions are stored in the computer-readable storage medium, the computer program instructions, when executed by a processor, causing the processor to perform the ultrasonic blood perfusion imaging method for a single blood vessel according to claim 1.
